(19) United States
(12) Patent Application Publication
(10) Pub. No.: US 2006/0201809 A1
(43) Pub. Date: Sep. 14, 2006

(54) Sample injection method using capillary plate

(75) Inventors: Naoya Endo, Kyoto-shi (JP); Tetsuo Ohashi, Kyoto-shi (JP); Shin Nakamura, Kyoto-shi (JP)

Correspondence Address: KANESAKA BERNER AND PARTNERS LLP, SUITE 300, 1700 DIAGONAL RD, ALEXANDRIA, VA, 22314-2848, US

(73) Assignee: SHIMADZU CORPORATION, Kyoto-shi (JP)

(21) Appl. No.: 11/369,814

(22) Filed: Mar. 8, 2006

(30) Foreign Application Priority Data
Mar. 9, 2005 (JP) 2005-065527

(51) Int. Cl. C07K 1/26

(52) U.S. Cl. 204/453

(57) ABSTRACT

In a sample injection method to a capillary plate, a first liquid is provided into a large-capacity reservoir and a small-capacity reservoir formed at a bottom of the large-capacity reservoir. A sample is dissolved in a second liquid having heavier specific gravity than the first liquid, and the sample dissolved in the second liquid is injected through the first liquid into the small-capacity reservoir.

### BACKGROUND OF THE INVENTION AND RELATED ART STATEMENT

The present invention relates to a method of analyzing extremely minute quantities of proteins and amino acids, drugs, and the like, in the fields such as biochemistry, molecular biology, clinical practice, and the like. In particular, the invention relates to a method of injecting a sample into each capillary channel of a capillary plate having plural capillary channels in order to perform separation of sample components in the respective capillary channels.

Such capillary plate can be used in capillary electrophoresis or liquid chromatography.

Electrophoresis devices have been used from the past when analyzing extremely minute quantities of proteins and amino acids, and the like. As a representative, there is a capillary electrophoresis device having capillary tubes. However, the handling of a device having capillary tubes is complicated. Therefore, a capillary plate having plural capillary channels formed inside a substrate has been proposed and used with the purpose of making the handling easier and also for acceleration of analysis and miniaturization of the device (see Patent Documents 1 and 2).

The capillary channels of the capillary plate serve as separation channels for electrophoresis or columns for liquid chromatography, and both ends are opened on the substrate surface. The openings on one end side serve as sample reservoirs for sample injection, and the samples are injected into the sample reservoirs in advance of analysis.

At the time of sample injection in capillary electrophoresis or liquid chromatography, first, the sample reservoirs are cleaned, and the samples are injected after removing all of the residual liquid, and then, the samples are introduced into the capillary channels from the sample reservoirs to perform analysis.

Patent Document 1: Japanese Unexamined Patent Publication No. 2002-310990

Patent Document 2: Japanese Unexamined Patent Publication No. 2003-166975

In capillary electrophoresis or liquid chromatography, the capillary parts or column parts often become in a high-temperature condition for improvement of its analytical performance. There is a problem that when a minute quantity of samples is injected into the sample reservoirs in that environment, the samples dry up in a short time.

Also, because of the above situation, there also is a problem that the quantity of samples cannot be reduced.

The present invention therefore has an object to suppress the drying of the samples so that even minute quantities of samples can be injected.

Other objects and advantages of the invention will be apparent from the following description of the invention.

### SUMMARY OF THE INVENTION

The present invention is a method of injecting a sample into each capillary channel of a capillary plate having plural capillary channels in order to perform separation of sample components in the respective capillary channels, wherein a large-capacity reservoir having a capacity to contain sample injection parts of plural capillary channels is provided on at least the sample injection side of the capillary plate, and small-capacity reservoirs for the respective sample injection parts of the respective capillary channels are provided on the bottom of that large-capacity reservoir. In the method, a first liquid is put into the large-capacity reservoir so as to fill the small-capacity reservoirs in advance of sample injection, and samples dissolved in a second liquid having heavier specific gravity than the first liquid are passed through the first liquid to be injected into the small-capacity reservoirs of the respective capillary channels.

By this, the samples enter into the small-capacity reservoirs in a manner so as to sink to the bottom of the first liquid in a state being dissolved in the second liquid. Also, in the small-capacity reservoirs, the samples are insulated from air by the first liquid, and drying can be prevented.

As the second liquid for dissolving the samples, a liquid having low viscosity and tending not to volatize is preferable. In the case of using water or a liquid having specific gravity near that as the first liquid, the second liquid can be constituted mainly by water and contain at least one selected from the group consisting of polyvalent alcohols, sugars, and other hydrophilic polymer compounds. These compounds are easily dissolved in water and also have high chemical stability. In the case when the samples are biopolymers such as amino acids or proteins and when performing separation by electrophoresis in the capillary channels, these compounds can keep the samples in a state suitable for analysis.

As polyvalent alcohols, bivalent alcohols and trivalent alcohols, for example, ethylene glycol, glycerol, pentaerythritol, propylene glycol, and mannitol, and the like, can be mentioned. As sugars, monosaccharides, and oligosaccharides and polysaccharides having plural of these condensed, are included, concretely, glucose, sucrose, dextran, and the like, can be mentioned. In the case when polyvalent alcohols are contained in the second liquid, they are contained preferably at 5˜80(w/v)%, more preferably 20˜60(w/v)% in the solution. In the case when sugars are contained in the second liquid, they are contained preferably at 5˜80(w/v)%, more preferably 20˜60(w/v)% in the solution.

The capillary plate in the present invention should have plural capillary channels on a substrate. One example of capillary channels is formed by forming fine grooves on the surface of one substrate and overlaying and bonding another substrate on its surface. Another example of capillary channels is capillary tubes, and the capillary plate in that case is made by arranging capillary tubes on a substrate and integrating them with the substrate.

The small-capacity reservoir can be formed as a cavity having smaller diameter than the large-capacity reservoir.

Also, the front end of the respective capillary channel can be opened on the bottom surface of the large-capacity reservoir, and the bottom surface of the large-capacity reservoir can be surface-treated so that only the periphery of the opening becomes hydrophilic and the outside of that becomes hydrophobic, whereby the small-capacity reservoir can be formed by the opening and its periphery.

In the conventional capillary electrophoresis or liquid chromatography, a certain amount of liquid was necessary in order not to let the samples dry when injecting into the separation mechanism. Therefore, because the present invention was made such that liquid is put into the large-capacity reservoir, and samples dissolved in a liquid having heavier specific gravity than that liquid are injected through that liquid into the small-capacity reservoirs of the respective capillary channels, the samples are insulated from air by the liquid in the large-capacity reservoir. Therefore stable dripping and injection of the samples without accompanying risk of evaporation of the samples in a high-temperature environment can be performed, and drying can be prevented. Also, by preventing drying of the samples, the quantity of the samples can be reduced.

Because the small-capacity reservoirs serve as the sample reservoirs, the samples can be injected stably even when the samples are minute quantities.

Also, because small-capacity reservoirs of plural capillary channels are provided inside the large-capacity reservoir, the operations of polymer packing and cleaning of the capillary channels, dripping of samples into the small-capacity reservoirs, and electrophoresis in the capillary channels can be performed simultaneously through the plural capillary channels, and improvement of operability and shortening of time can be accomplished.

### BRIEF DESCRIPTION OF THE DRAWINGS

FIGS. 1(A)-1(D) are drawings showing one example of a capillary plate to which the present invention is applied, wherein FIG. 1(A) is a plan view of the capillary channels, FIG. 1(B) is an enlarged plan view of the sample reservoir (small-capacity reservoir) part on the cathode end, FIG. 1(C) is a perspective view of the cathode end, and FIG. 1(D) is a sectional view of the cathode end taken along line 1(D)-1(D) in FIG. 1(B).

FIG. 2 is a sectional view of the end on the cathode side of another capillary plate.

FIG. 3 is a sectional view of the end on the cathode side of yet another capillary plate.

FIG. 4 is a sectional view of the end on the cathode side showing dripping of a sample in water.

FIG. 5 is a waveform graph showing one example of the results of electrophoretic separation.

### DETAILED DESCRIPTION OF PREFERRED EMBODIMENTS

Below, a working example of the present invention, which uses an MEMS (Micro Electro Mechanical System) capillary plate as electrophoresis component, is explained in detail while referring to the drawings.

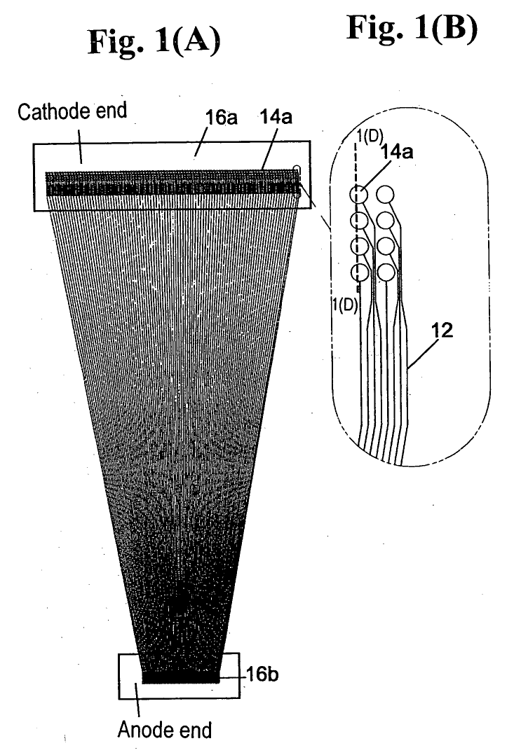

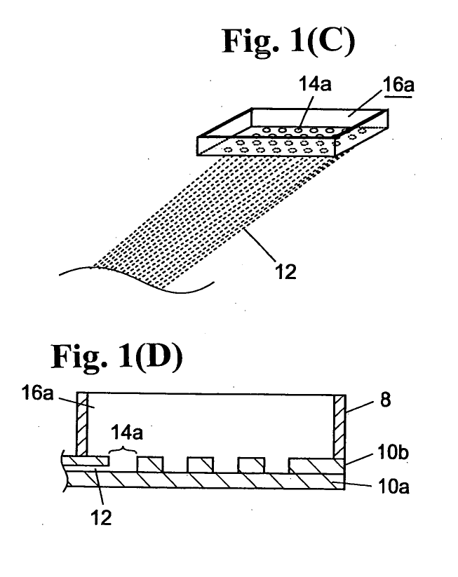

FIG. 1(A) is a plan view of the capillary channels in an electrophoresis component consisting of a capillary plate, FIG. 1(B) is an enlarged plan view of the sample reservoir (small-capacity reservoir) part on the cathode end, FIG. 1(C) is a perspective view of the cathode end, and FIG. 1(D) is a sectional view of the cathode end taken along line 1(D)-1(D) in FIG. 1(B).

The electrophoresis component has a pair of plate members 10a, 10b bonded together. On one plate member 10a, plural, for example 384, separation channels 12 consisting of capillary channels are formed, and they are arranged so as not to intersect with each other.

One end (cathode end) of each separation channel 12 is connected to a small-capacity reservoir 14a which is a sample reservoir opened on the substrate surface, and on the substrate surface, a large-capacity reservoir 16a having a size containing all the small-capacity reservoirs 14a is formed to be surrounded by a wall 8. The other end (anode end) of each separation channel 12 is opened so as to be connected to a common reservoir 16b formed on the substrate surface.

The width of the separation channel 12 is 10˜1000 μm, preferably 50˜130 μm, and the depth is 10˜1000 μm, preferably 20˜60 μm. On the other plate member 10b, through-holes are formed in positions corresponding to the two ends of the separation channel 12. The through-hole on one end side is the small-capacity reservoir 14a, and the diameter of the small-capacity reservoir 14a is 10 μm˜3 mm, preferably 50 μ˜2 mm, and it is set to a size suitable for injecting several 10 nL˜several μL of sample. Both plate members 10a and 10b are affixed together with the separation channels 12 on the inside to become a single plate member.

Formation of the separation channels 12 on the plate member 10a can be done by lithography and etching (wet etching or dry etching). Formation of the through-holes on the plate member 10b can be done by a method such as sand blasting or laser drilling.

The entire area of the small-capacity reservoirs 14a is covered by the large-capacity reservoir 16a, and as in FIG. 1(C) showing the perspective view, all the small-capacity reservoirs 14a are provided inside the large-capacity reservoir 16a, and they are connected with the reservoir 16a. The reservoir 16b on the other end side also covers the area where the openings on the other end side of all the separation channels 12 are disposed, and the openings on the other end side of all the separation channels 12 are connected with the reservoir 16b.

As for the material of the plate members 10a, 10b constituting the substrate, quartz glass or borosilicate glass, resin, or the like, can be used, and a transparent material is selected in the case when the components separated by phoresis are detected optically. In the case when using a detecting means other than light, the material of the plate members 10a, 10b is not limited to one that is transparent.

The inner wall of the small-capacity reservoir 14a may be made hydrophilic, and the bottom surface of the large-capacity reservoir 16a or from the bottom surface to the inner wall surface may be made hydrophobic.

As for the surface treatments for such hydrophilic and hydrophobic properties, various methods can be mentioned. For example, in the case of using a glass plate as the plate member, the hydrophilic property can be given by acid treatment, and the hydrophobic property can be given by coating with resin, processing with fluorine resin or treating with silane coupling agent, or the like.

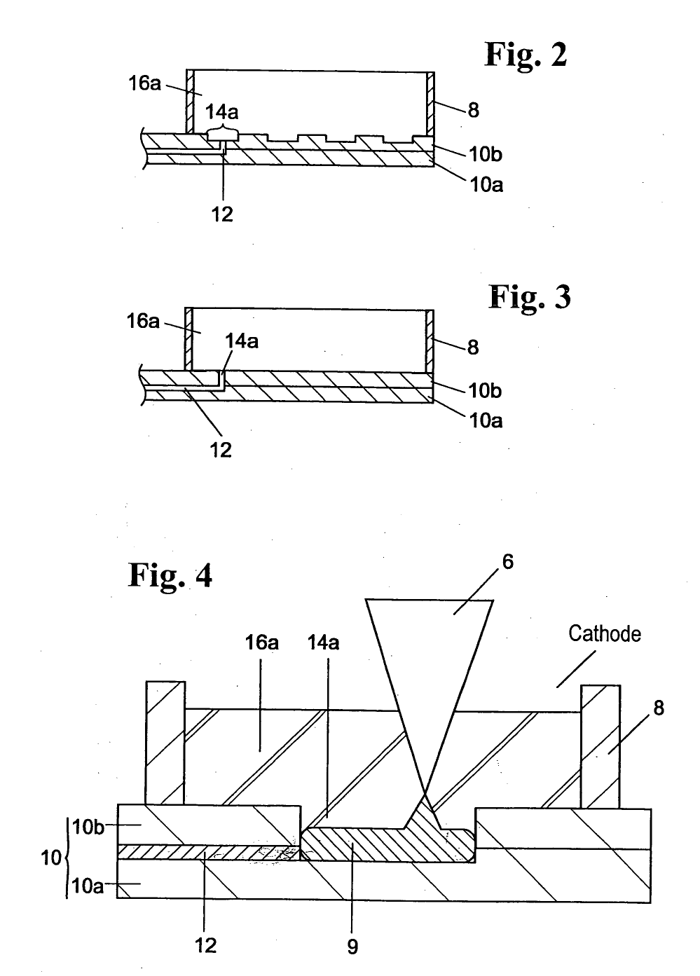

FIG. 2 shows a sectional view on the cathode side of another capillary plate. The small-capacity reservoir 14a is formed as a cavity on the surface side of the plate member 10a, and it is connected at the bottom with the separation channel 12. Plural small-capacity reservoirs 14a are covered by a large-capacity reservoir 16a, and they are formed on the bottom surface of the large-capacity reservoir 16a.

FIG. 3 shows a sectional view on the cathode side of yet another capillary plate. The small-capacity reservoir 14a is formed as an opening having a size about the same extent as the separation channel 12.

In either of these capillary plates shown in FIG. 2 or FIG. 3, surface treatment may be applied so that the small-capacity reservoir 14a and a narrow range of the periphery of the opening of the small-capacity reservoir 14a on the bottom surface of the large-capacity reservoir 16a become hydrophilic, and the outside of that becomes hydrophobic. By this, the injected sample comes to be held in the part applied with hydrophilic treatment, and that hydrophilic area becomes the small-capacity reservoir. The size of that hydrophilic area is set to a size suitable for the quantity of sample held to become several 10 nL˜several μL.

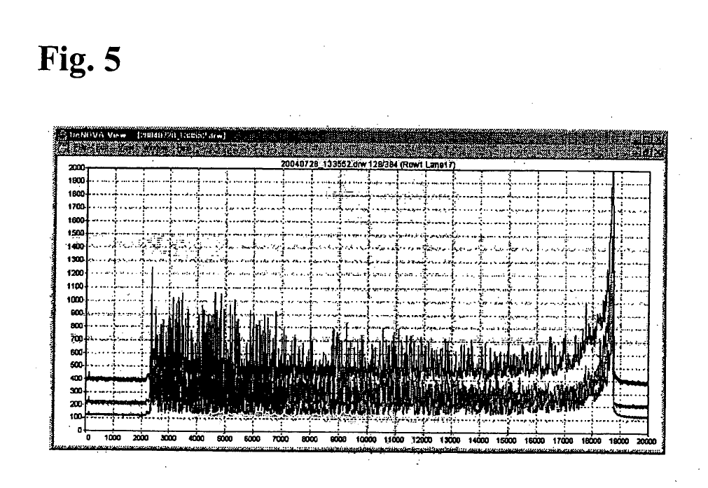

Next, the sample injection operation in the capillary plate in FIGS. 1(A)-1(D) is explained while referring to FIG. 4.

(1) The capillary plate is kept in a constant-temperature state of 50° C.

(2) The large-capacity reservoir 16a on the cathode side is filled with pure water, for example Milli-Q water which is ultra-pure water, and polymer is packed into all the separation channels 12 by pressurizing by syringe from the anode side.

(3) Because the polymer flowing out from the separation channels 12 to the small-capacity reservoirs 14a diffuses in the pure water of the large-capacity reservoir 16a, the water and the polymer inside the reservoirs 14a, 16a are drawn by a suction nozzle, and the insides of the reservoirs 14a, 16a are cleaned.

(4) After cleaning the insides of the reservoirs 14a, 16a, buffer solution is filled into the cathode-side reservoir 16a and the anode-side reservoir 16b, voltage is applied between the two reservoirs 16a, 16b to perform pre-separation, and ions of impurities in the polymer are caused to move toward the anode electrode or the cathode electrode. The applied voltage is, for example, 125V/cm, and the application time is 5 minutes.

(5) The buffer solution in the cathode-side reservoir 16a is drawn, and the inside of the reservoir 16a is cleaned, and then the inside of the reservoir 16a is cleaned with pure water, for example Milli-Q water which is ultra-pure water.

(6) After that, samples 9 are dripped successively or in units of plurality drops bypipetter 6 into each small-capacity reservoir 14a of the reservoir 16a filled with pure water. Dripping of the samples is performed by lowering the front end of the pipetter 6 to near the small-capacity reservoir 14a. The samples 9 are in a state dissolved in a solution of ethylene glycol, or the like, having greater specific gravity than water, and a minute quantity of sample, i.e. a volume of about 0.1μseveral μL, is dispensed. Also at this time, the capillary plate is maintained in a constant-temperature state of 50° C.

(7) A cathode electrode is inserted into each small-capacity reservoir 14a, and voltage is applied between it and the anode electrode to perform sample injection into the channel 12. The applied voltage for sample injection is, for example, 50V/cm, and the application time is 40 seconds.

(8) After drawing the pure water in the reservoir 16a as well as the remaining samples in the small-capacity reservoirs 14a and cleaning, the insides of the reservoirs 14a, 16a are filled with buffer solution.

(9) A cathode electrode is inserted into the reservoir 16a, and phoresis voltage is applied between it and the anode electrode to perform electrophoresis separation and signal detection of the sample. The applied voltage for electrophoresis separation is suitably 70˜300V/cm, for example, 125V/cm.

The electrode may be provided in advance respectively in the reservoirs 16a, 16b, and it also may be inserted separately. Also, on the sample injection side, the cathode electrode may be provided in each small-capacity reservoir 14a, and it also may be inserted separately.

The measurement conditions are as follows.

The DNA sample was prepared using the BigDye v3.1 reagent kit for cycle sequencing (manufactured by Applied Biosystems Corporation). The template DNA was 12.5 ng/μL of pUC18 plasmid DNA (manufactured by Toyobo Corporation), and synthetic primer was used for the primer.

The other conditions followed the kit handling instructions, and a standard product was obtained by performing ethanol precipitation processing and then drying and hardening.

The above dry standard product was dissolved using sample preparation solution containing 50% ethylene glycol, 0.4 mM Tris-HCl (pH 8.0) and 0.04 mM EDTA such that the dry standard product to sample preparation solution became 1:8, and sample solution to supply to the sequencer was prepared. Each sample solution was filled by pipetter into the sample reservoir 14a formed on the capillary plate shown in FIGS. 1(A)-1(D). The upper surface of the sample reservoir 14a was filled with water, and the sample solution was dripped from directly above with the tip of the pipetter at a distance of about 0.5 mm from the opening of the sample reservoir 14a.

An example of an electrophoresis pattern made under the above conditions is shown in FIG. 5.

The electrophoresis pattern is the result of projecting excited light on the DNA sample separated by electrophoresis in the detection part and detecting its fluorescence. The horizontal axis represents the scan number when scanned with the excited light, and it corresponds to the time. The vertical axis is the fluorescence strength. The graph includes four waveforms corresponding to the four kinds of bases, A (adenine), G (guanine), C (cytosine), and T (thymine).

By the results of the working example, the signal strength of fluorescence detection of each peak separated by phoresis is great, and it is clear that it indicates a good separation state.

The sample injection method of the present invention can be used in the fields such as biochemistry, molecular biology, and clinical practice, and the like.

The disclosure of Japanese Patent Application No. 2005-065527 filed on Mar. 9, 2005 is incorporated herein.

While the invention has been explained with reference to the specific embodiments of the invention, the explanation is illustrative, and the invention is limited only by the appended claims.

1. A sample injection method, comprising:
providing a first liquid into a large-capacity reservoir and a small-capacity reservoir formed at a -bottom of the large-capacity reservoir,
dissolving a sample in a second liquid having heavier specific gravity than the first liquid, and
injected the sample dissolved in the second liquid through the first liquid into the small-capacity reservoir.

2. A sample injection method according to claim 1, wherein a capillary plate is prepared such that the large-capacity reservoir includes a plurality of small-capacity reservoirs at the bottom, which communicate with respective capillary channels.

3. A sample injection method according to claim 1, wherein the small-capacity reservoir is formed as a cavity having a smaller diameter than the large-capacity reservoir.

4. A sample injection method according to claim 1, wherein a front end of each capillary channel is opened on the bottom of the large-capacity reservoir, and the bottom of the large-capacity reservoir is surface treated so that only a periphery of the opening becomes hydrophilic and an outside of the periphery becomes hydrophobic so that the small-capacity reservoir is formed by the opening with hydrophilic property.

5. A sample injection method according to claim 4, wherein said small-capacity reservoir is surface treated to become hydrophilic.

6. A sample injection method according to claim 1, wherein the sample is injected into a capillary channel to perform separation of sample components in the capillary channel.

7. A sample injection method according to claim 1, wherein the second liquid with the sample is placed into a pipetter, and the pipetter is immersed into the first liquid near the small-capacitor reservoir and the second liquid is supplied to the small-capacitor reservoir through the pipetter.

8. A sample injection method according to claim 7, wherein the large and small-capacitor reservoirs are heated above a room temperature.

9. A sample injection method according to claim 2, wherein the capillary channels are used for electrophoresis.

10. A sample injection method according to claim 2, wherein said large-capacity reservoir with the small-capacity reservoirs is provided on one side of the capillary plate, and another reservoir is provided on the other side of the capillary plate with a plurality of capillary channels formed therebetween.

11. A sample injection method according to claim 10, wherein electrodes are inserted into the small-capacity reservoirs and the another reservoir, and voltage is applied therebetween.
